(19) United States
(12) Patent Application Publication
(10) Pub. No.: US 2011/0256155 A1
(43) Pub. Date: Oct. 20, 2011

(54) METHODS TO MOLECULARLY CHARACTERIZE CIRCULATING TUMOR CELLS

(75) Inventors: Jiaqi Huang, Potomac, MD (US); Haifeng Bao, Rockville, MD (US); Philip Brohawn, Jefferson, MD (US); Patricia Burke, Ranson, WV (US); Xiaoru Chen, Sterling, VA (US); Theresa Lavallee, Rockville, MD (US); Yihong Yao, Boyds, MD (US)

(73) Assignee: MEDIMMUNE, LLC, Gaithersburg, MD (US)

(21) Appl. No.: 13/088,751

(22) Filed: Apr. 18, 2011

Related U.S. Application Data
(60) Provisional application No. 61/325,042, filed on Apr. 16, 2010.

(51) Int. Cl. A61K 39/395; A61P 35/00; C12Q 1/68

(52) U.S. Cl. 424/174.1; 435/6.1; 435/6.14; 435/6.12

(57) ABSTRACT

The invention relates to a rapid, sensitive method to obtain a gene expression profile from a target cell population in a blood sample. The target cells can be circulating tumor cells. Disclosed are methods and kits for obtaining such profiles.

### BACKGROUND OF THE INVENTION

Circulating tumor cells (CTCs) are cancer cells circulating in the peripheral blood that have been shed from either a primary tumor or its metastases. The raw number of CTCs in whole blood of cancer patients has clinical relevance with respect to patient prognosis. Additionally, interest exists in characterization of these isolated CTCs on a molecular level.

A variety of systems that seek to isolate cells exist, for example the CELLSEARCH isolation system (Veridex LLC, Warren, N.J., USA), which has been used to isolate CTCs. However these systems present difficulties with regard to performing molecular characterization of CTCs. For example, CELLSEARCH relies on collection of whole blood (WB) into CellSave tubes which contain EDTA, as do conventional blood tubes, along with a “cell preservative” or fixation agent. CTCs are then captured using magnetic nanoparticles conjugated to an antibody specific for a cell surface marker present on epithelial cells.

One difficulty with existing cell isolation systems with regard to gene expression profiling lies in an inability to enrich for CTCs while adequately depleting leukocytes. As a result, the CTC-enriched fractions typically contain sufficient numbers of leukocytes so as to interfere with and sometimes completely confound CTC-specific gene expression profiling. Another difficulty in using such systems to generate robust results is the decreased assay sensitivity due to the cell preservative fixation reagents used in standard blood collection tube, which tend to interfere with efforts to ascertain mRNA expression.

### BRIEF SUMMARY OF THE INVENTION

In a first aspect, a rapid, sensitive method to obtain a gene expression profile from a target cell population in a blood sample comprises: (a) enriching the blood sample in target cells to obtain an enriched target cell sample; (b) treating the enriched sample with a protease; (c) extracting and purifying RNA from the sample; (d) reverse transcribing the purified RNA to obtain a plurality of cDNAs; and (e) analyzing the cDNAs with an amplification technique to obtain a target cell gene expression profile comprising expression levels of a plurality of mRNAs of interest, wherein the target cell population represents less than 10% of the total cells in the blood sample.

In a further aspect, a method of treating a patient having a tumor comprises (a) obtaining a circulating gene expression profile from the patient by the method of the first aspect, and (b) performing a medical treatment on the patient based on the circulating tumor cell gene expression profile.

In another aspect, a kit comprises an antibody or functional fragment thereof specific for a cell surface marker found on tumor cells, and primers adapted to amplify at least one cDNA, wherein the at least one cDNA comprises at least one cDNA not normally expressed in leukocytes.

### BRIEF DESCRIPTION OF THE DRAWINGS

FIG. 1 shows expression levels of various markers in different tumor cells measured as described herein. Briefly, 10, 50 and 100 MG63 and SKLMS cells were spiked into EDTA or CellSave tubes. The samples were incubated with 100 μl proteinase K buffer, followed by 300,al Trizol LS. RNA was isolated from the treated lysate using Zymo mini RNA spin columns. Superscript 111 cDNA synthesis utilizing random hexamer priming was performed. Target genes of interest were then pre-amplified using established methodology and reagents. The resulting volume of pre-amplified eDNA was diluted resulting in ¼ of the cDNA being profiled on the Fluidigm Biomark 48.48 Dynamic Array following the manufacturer's established protocol. The PDGFRα expression levels were calculated using the delta method and utilizing 18s as the reference gene.

FIG. 2 shows the results of qualitative measurement of expression of several genes in CTC samples from cancer patients. 7.5 ml of whole blood from cancer patients were collected in CellSave tubes. Samples were processed in the CellSearch System according to manufacturer's instructions. The collected cells were treated with proteinase K buffer, RNA was isolated, and eDNA was generated, according to the process described throughout the poster. Resulting eDNA was concentrated into a set volume utilizing magnetic beads. The cDNA of the target genes of interest were pre—amplified. The pre-amplified cDNA was then diluted and profiled on the Fluidigm Biomark 48.48 Dynamic Array following the manufacturer's established protocol.

FIG. 3 shows quantitative measurement of specific target gene expression in the CTC-containing whole blood samples from pancreatic cancer patients. The KRT20 gene is a specific epithelial gene and is consistently expressed in CTC samples from the two pancreatic cancer patients in our test panel. KRT20 expression level in individual samples was used as reference gene to calculate the relative expression level of the other genes examined, such as CECAM5, DLL4, and EPHA2.

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the terms “a” or “an” mean “at least one” or “one or more.”

As used herein, the term “antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies can include a complete immunoglobulin or fragment thereof, which immunoglobulins include the various classes and isotypes, such as IgA, IgD, IgE, IgG1, IgG2a, IgG2b and IgG3, IgM, etc. Fragments thereof can include Fab. Fv and F(ab′)2, Fab′, and the like. In addition, aggregates, polymers, and conjugates of immunoglobulins or their fragments can be used where appropriate so long as binding affinity for a particular polypeptide is maintained.

As used herein, the term “antibody binding composition” means a molecule or a complex of molecules that comprises one or more antibodies, or fragments thereof, and derives its binding specificity from such antibody or antibody fragment. Antibody binding compositions include, but are not limited to, (i) antibody pairs in which a first antibody binds specifically to a target molecule and a second antibody binds specifically to a constant region of the first antibody; a biotinylated antibody that binds specifically to a target molecule and a streptavidin protein, which protein is derivatized with moieties such as molecular tags or photosensitizers, or the like, via a biotin moiety; (ii) antibodies specific for a target molecule and conjugated to a polymer, such as dextran, which, in turn, is derivatized with moieties such as molecular tags or photosensitizers, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as molecular tags or photosensitizers, or polymers containing the latter.

As used herein, the term “enriching” means increasing the percentage of target cells present in a sample in relation to other cells in that sample. For example, enriching a sample in circulating tumor cells can include increasing the percentage of circulating tumor cells relative to leukocytes.

As used herein, the term “fixation” with regard to a blood sample refers to subjecting the blood sample to a cell preservative, such as paraformaldehyde, formaldehyde, or the like, in an amount effective to increase the stability of the blood sample.

As used herein, the term “kit” refers to any delivery system for delivering materials. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents can be delivered to the intended recipient together or separately.

As used herein, the term “rapid” refers to the ability of an assay or method to be completed within one conventional working day, namely within about eight hours.

As used herein, the term “sensitive” refers to the ability of an assay to detect expression of a cDNA of interest from a small number of cells within a background of a much larger number of cells. In some embodiments, the sensitivity of the assay is at least equivalent to the ability to detect the expression of a marker such as PDGFRα in as few as five tumor cells spiked into a background environment containing approximately 1000 or more contaminating leukocytes obtained from normal blood.

### B. METHODS AND KITS FOR OBTAINING A CIRCULATING TUMOR CELL GENE EXPRESSION PROFILE

Provided herein are methods which allows the accurate profiling of select transcripts of interest in target cells in the blood, such as circulating tumor cells. In one aspect, the method overcomes the difficulties presented by the CELLSEARCH isolation system and other similar systems while retaining the blood sample stability provided having a cell preservative in a blood collection tube. Typically, in a clinical setting, the traditional blood collection tube containing EDTA requires equires rapid handling before degradation of cells, making it difficult or impossible to effectively analyze the gene expression profile before undesired events such as cell lysis and concomitant digestion of mRNAs. More particularly, provided herein is a method to obtain a gene expression profile from a target cell population in a blood sample comprises: (a) enriching the blood sample in target cells to obtain an enriched target cell sample; (b) treating the enriched sample with a protease; (c) extracting and purifying RNA from the sample; (d) reverse transcribing the purified RNA to obtain a plurality of cDNAs; and (e) analyzing the cDNAs with an amplification technique to obtain a target cell gene expression profile comprising expression levels of a plurality of mRNAs of interest, wherein the target cell population represents less than 10% of the total cells in the blood sample.

Also provided herein are methods to qualitatively measure target genes of interest in a discrete target cell population in a blood sample comprising (a) enriching the sample in target cells to obtain an enriched target cell sample; (b) treating the enriched sample to digest proteases; (c) extracting nucleic acids from the sample using organic extraction; (d) purifying RNA from the sample; (e) performing reverse transcription on the purified RNA to obtain a plurality of cDNAs; and (f) analyzing the cDNAs to determine the level of expression of at least one target gene of interest, wherein the target cell population represents less than 10% of the total cells in the blood sample. Typically, the target gene is a specific therapeutic target. In some embodiments, the target gene is DLL4, EphA2, Her3, PGDFRα, CEACam5, or some combination thereof. The target cell population can be the circulating tumor cell population.

The methods described herein permit detection of the expression of genes from quite small cell populations. The methods disclosed herein have resulted in successful quantitative gene expression analysis on the marker such as PDGFRα using as few as five tumor cells spiked into a background environment containing approximately 1000 or more contaminating leukocytes obtained from normal blood. Gene expression analysis was also successful at the two cell level without interference from any residual leukocyte background. The methods have further been found to provide qualitative and quantitative gene expression profiles from blood samples from cancer patients.

It is expected that such gene expression profiles will be useful in characterizing the molecular profile of an individual patient's tumor and thus allow an individualized approach for therapy using biologics as well as other therapeutics. This is especially desirable because the mutations involved in cancer generally result in substantial differences in cancer cell populations among individuals, thereby challenging convention diagnosis and treatment.

Furthermore, a method using circulating tumor cells from the peripheral blood is highly valued because such blood is easy to obtain, eliminates the need for often difficult and expensive procedures necessary for tumor biopsy, and can be readily performed repeatedly to assess any changes in the molecular profile of a tumor during treatment. For example, if a patient's CTC profile indicated the presence of a particular molecular marker, then this patient would be a candidate for treatment using a biologic or corresponding therapeutic agent that is known or suspecting of being effective against cancer cells expressing that marker. As disclosed herein, the expression of such markers can be readily evaluated over the course of treatment.

The methods and kits described herein can be employed in conjunction with the diagnosis, prognosis, and/or treatment of any suitable variety of cancer and/or tumor. Exemplary types of tumors and cancers include, but are not limited to, adenoid, carcinoma such as cystic carcinoma, lymphoma, blastoma (including medulloblastoma and retinoblastoma), sarcoma (including liposarcoma, spindle cell sarcoma, and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), cancers of the adenoid, lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer such as adenoid cystic carcinoma.

In one aspect, a blood sample is a quantity of whole blood (for example, 7.5 ml) that has been collected from an individual using any suitable means, such as a blood collection tube. In one aspect, such a blood collection tube contains EDTA, and it can also contain a cell preservative. In one embodiment, a CellSave tube is used. The methods as described herein are sensitive and thus advantageously require only a relatively small amount of peripheral blood, such no more than 5 ml, or no more than 10 ml, thereby reducing or eliminating requirements for taking large blood samples, or for tumor biopsy in another embodiment, larger quantities of blood, such as 10 to 50 ml, or 20 to 100 ml, or 50 to 500 ml, are employed in order to possibly obtain a larger population of target cells for analysis. In still another embodiment, relatively smaller quantities of blood are employed, such as 5 to 7.5 ml, or 3 to 5 ml, or 1 to 3 ml, or 100 microliters to 1 ml.

In one aspect, the blood sample has been subjected to contact with a cell preservation agent and/or a fixative. Thus, the blood sample is relatively stable compared to such a sample in a conventional EDTA collection tube. A sample in a conventional EDTA tube must normally be processed within 48 hours, whereas a sample contacted with a cell preservation agent and/or a fixative, for example a sample collected in a CellSave tube, can be stable for up to 96 hours following collection.

The target cells in the blood sample (such as CTCs) are normally a minority of the cells in the blood sample, and frequently are a very minor fraction of the cells in the sample. For example, the target cells can be less than 10%, or than 5%, or than 1%, or than 0.5%, or than 0.1%, of the total cells in the blood sample.

The collected blood is enriched in target cells using any suitable means. The target cells are optionally circulating tumor cells. In one aspect, the enrichment is accomplished by an antibody or functional fragment thereof specific for a cell surface marker found on the target cell population. In some embodiments, the antibody would be directed to a cell surface marker for epithelial cells or a particular tumor type. In one embodiment, such an antibody is specific for EpCAM.

In some embodiments, the enrichment can be accomplished when an antibody is conjugated to a magnetic bead or particle, which facilitates the separation of the target cell population from other cells. In one aspect, target cells are captured for enrichment via a capture antigen that is attached to a magnetic particle for separation. Capture antigens can be any cell surface antigen that is differentially expressed on the target cells relative to other circulating cells, such as leukocytes and red blood cells. In one aspect, capture antigens are cell surface receptors that are expressed exclusively on the target cells, or that are over expressed on the target cells relative to other cells in circulation. Magnetic particles are provided that have attached an antibody composition specific for such capture antigen. These magnetic particles can be mixed with a blood sample suspected of containing the target cells under conditions that allow the antibody composition to form a stable complex with capture antigens whenever present in the sample. A magnetic field is then applied to the magnetic particles to immobilize them during a washing step to remove un-complexed material, or transport captured cells away from the un-complexed material. In either case, an enriched target cell sample is formed that comprises a population of cells enriched for those having the capture antigen.

An exemplary enrichment method relies on the CELLSEARCH isolation system. Other suitable means of enriching a sample in circulating tumor cells are known to those in the art, for example those of the Adna Test kit of AdnaGen AG, and those described in U.S. Pat. No. 7,537,938, incorporated herein by reference.

Previously, it was challenging to extract and purify useful RNA from blood collected in a CellSave tube due to the presence therein of the cell preservative, resulting in fixation of the contents of the tube. It has been found that treating the enriched sample with a protease can overcome the impact of fixation on RNA isolation. In one aspect the protease is a serine protease. Optionally, the protease is proteinase K.

RNA in the sample can be extracted and purified from the sample by any suitable means. The extraction of the RNA can be performed following the protease treatment and/or in conjunction with the protease treatment. In one embodiment, the enriched blood sample is contacted with a protease, together with optionally a detergent such as SDS, optionally a reducing agent such as DTT, optionally a chelator such as EDTA, and optionally an RNAse inhibitor, and then incubated, for example for one hour at 37° C. Other reagents and incubation conditions can be used, for example two hours at room temperature. Thereafter, the sample can be centrifuged and the supernatant containing the RNA collected and mixed with TRIZOL. Other techniques for extracting and purifying the RNA are known in the art, for example those employing phenol and chloroform. Optionally, at this point the samples can be frozen for storage, such as at −80° C., or −70° C., or in liquid nitrogen, or under other suitable conditions as known in the art.

If frozen, the samples comprising the RNA are typically thawed completely at room temperature to continue the RNA purification using any suitable methods. For example, an amount of chloroform, such as 80 μl, can be added to each sample and the samples mixed thoroughly, incubated, and then centrifuged to separate the aqueous phase containing RNA. Thereafter, 0.8 volumes of 100% ethanol can be added prior to RNA purification on, for example, a micro-scale spin column.

In one aspect, the plurality of mRNAs of interest comprises at least one mRNA not normally expressed in leukocytes, and optionally most or all the cDNAs are not normally expressed in leukocytes. Leukocyte contamination of samples enriched in CTC target cells was found to limit the usefulness of analysis to those cDNAs not normally expressed in leukocytes, otherwise the contribution from the relatively small numbers of CTCs becomes difficult to isolate from that of the leukocytes. In one aspect, the purified RNA also contains other RNA such as 18S ribosomal RNA, which can be a useful internal control as known in the art.

The RNA obtained from the enriched sample is subjected to reverse transcription using any suitable means, thus obtaining a plurality of cDNAs corresponding to the target cell gene expression profile. In one aspect, a random hexamer priming protocol is employed, and the cDNAs are cleaned of RNA and concentrated using methods known in the art. The cDNA can be obtained by other than random hexamer priming, for example by primers specific to mRNAs of interest.

The plurality of cDNAs is then analyzed using an amplification technique by any suitable means, thus obtaining a circulating tumor cell gene expression profile comprising expression levels of a plurality of mRNAs of interest. Amplification aids in the detection of target expression from small numbers of cells.

In one aspect, the amplification technique uses the cDNAs as templates for amplification together with primers corresponding to the genes of interest. Such primers can be commercially available.

Generally, suitable amplification techniques incorporate a polymerase chain reaction (PCR). In one embodiment the analysis is by real-time PCR. Another embodiment incorporates analysis using a dynamic array. In an exemplary method of analyzing the cDNAs, a pre-amplification reaction using the Applied Biosystems protocol and reagents (ABI, part#4391128) can be employed. Primers for the cDNAs selected for the profile are known in the art and commercially available, for example from Applied Biosystems. Applied Biosystems 20×TAQMAN assays for defined targets can be run on the Fluidigm Biomark 48.48 Dynamic Array according to the manufacturer's protocol, with initial data analysis utilizing the Fluidigm Gene Expression Analysis software, and subsequent analysis carried out in conventional computer spreadsheet software. Other suitable methods of amplification and analysis are known to those of skill in the art.

In one aspect, a kit of materials is provided comprising an antibody or functional fragment thereof specific for a cell surface marker found on target cells, and primers adapted to amplify at least one eDNA, wherein the at least one eDNA comprises at least one cDNA not normally expressed in leukocytes. The kit optionally also includes buffers, preservatives, and/or other reagents known in the art. In an embodiment, the cell surface marker is a marker for epithelial cells.

Using the methods described herein, quantitative gene expression analysis was successfully performed on the marker PDGRFα using as few as five tumor cells spiked into a background environment containing approximately 1000 or more contaminating leukocytes obtained from normal blood using the CELLSEARCH system. The methods have further been found to provide qualitative and quantitative gene expression profiles from blood samples from cancer patients.

The procedure can potentially be used to expand the understanding of the biology of CTCs and their potential role in metastasis, and to potentially improve patient management. For example, therapies can be tailored to individuals based on the CTC gene expression profile of that individual, so that therapies expected to act on a particular cancer can be provided and others avoided, thereby reducing treatment costs and potentially reducing side effects. Furthermore, due to the ease of collecting the small volume of blood required, profiles can be obtained from a patient over a course of treatment in order to adjust therapies over time.

Examples of potential genes of interest include, but are not limited to, MUC-1, EPCAM, TACSTD2, MGB1, KRT19 KRT20, S100A16, AGR2, ASGR2, PDGFRα, CEACAM5, EphA2, Dll4, EGFR, HER2, and HER3. Their sequences are known in the art. Other targets that can be desirable for inclusion in the described analysis include housekeeping genes that could serve as internal controls and/or markers for leukocytes that could allow for the detection of levels of leukocyte contamination.

### C. EXAMPLES

### I. General Methodology

Unless otherwise noted, the subsequent Examples employed the following methods.

A 7.5 ml blood sample was collected from an individual into a CellSave tube. The blood sample was then combined with 6.5 ml of buffer from the Profile Kit (Veridex). Samples were then centrifuged for 10 min at 800 g at room temperature and loaded onto the AutoPrep of the CELLSEARCH System. The samples were enriched in CTCs by the use of a ferrofluid coated with antibodies targeting Epithelial Cell Adhesion Molecule (EpCAM) antigen to select tumor (epithelial) cells. The samples (˜900 μl) were then placed on a MagCellect Magnet for 10 minutes, after which the supernatant was removed/discarded.

Blood samples were contacted with 100 μl of Proteinase K digestion buffer mix (Proteinase K (2 μl/100 μl), EDTA (1.02 mM), SDS (0.0051 g/ml), RNAaseOUT (500 U), DTT (0.612 mM)) and incubated for 1 h in a 37° C. water bath. The samples were then centrifuged for 5 min at 13,000 g. The supernatant was collected and 300 μl of TRIZOL reagent was added. The samples were then frozen at −80° C. until processed.

Samples removed from the freezer were thawed at room temperature. After each sample thawed completely, 80 μl of chloroform was added to each sample and the samples were mixed thoroughly by inverting several times and brief vortexing. Samples were then incubated on the benchtop for 5 minutes, followed by spinning in a microcentrifuge at 13,000 g for 5 minutes. The upper aqueous phase was removed and placed in a new 1.5 mL RNase free microcentrifuge tube. The volume was measured and 0.8 volumes of 100% ethanol were added. Samples were mixed thoroughly by pipetting up and down several times and 700 μl was then transferred to a Zymo Spin IC Column from the Zymo ZR RNA MicroPrep Isolation Kit (Zymo Research, cat #R1060). Samples are then centrifuged at 13,000 g for 30 seconds. Flow through was discarded and the column returned to the collection tube. Any remaining sample ethanol mixture was applied to the column and spun again at 13.000 g for 30 seconds. Flow through was again discarded and the column returned to the collection tube. 400 μl of RNA prep buffer from the Zymo kit was then added to each column. Samples were then centrifuged at 13,000 g for 1 minute. Flow through was discarded and the column was placed back into the collection tube. 800 μl of RNA Wash Buffer from the Zymo kit was then applied to the column and samples were spun at 13,000 g for 30 seconds. This wash was then repeated with a 400 μl of RNA Wash Buffer. Samples were then spun at 13,000 g for two minutes in an empty collection tube to ensure complete drying of columns. Sample columns were then transferred to a labeled RNase-free microcentrifuge tube. A 7 μl volume of RNase free water was added to each column to ensure a full 6 μl of recovery. Samples were spun at 10,000 g for 30 seconds to elute RNA from the columns.

The full 6 μl of RNA obtained from the column above was used to generate cDNA utilizing the SUPERSCRIPT 10 μl kit from Invitrogen, following the manufacturer's random hexamer priming protocol. Following cDNA generation, samples were diluted to 80 μl with RNase-free H₂O, and were cleaned and concentrated utilizing 144 μl of Agencourt RNA Clean bead reagent following the manufacturer's protocol (Beckman Coulter Genomics, Product#A29168). The cDNA was re-suspended in 10 μl of RNase-free H₂O.

The 10 μl of cDNA obtained as described above was utilized to run a 40 μl pre-amplification reaction, using the Applied Biosystems protocol and reagents (ABI, part#4391128), with 5 μl of the resulting reaction mix then diluted to 10 μl with RNase free water. Primers were also obtained from Applied Biosystems. This diluted sample and Applied Biosystems 20×TAQMAN assays for defined targets were then run on the Fluidigm Biomark 48.48 Dynamic Array according to the manufacturer's protocol. Initial data analysis was carried out utilizing the Fluidigm Gene Expression Analysis software with subsequent analysis carried out using MICROSOFT EXCEL.

### 2. Sensitivity and Reliability of the Assay without a Leukocyte Back Round

Quantities of 10, 50, and 100 MG63 (PDGFRα higher expressor) and SKLMS (PDGFRα medium expressor) cells were spiked into tubes containing EDTA or CellSave tubes (which contain EDTA and cell preservative). The samples were incubated with 100 μl Proteinase K buffer, followed by 300 μl TRIZOL LS. RNA was isolated from the treated lysate using Zymo mini RNA spin columns. Superscript III cDNA synthesis utilizing random hexamer priming was performed. Target genes of interest were then pre-amplified using the Life Technologies methodology and reagents. The cDNA was divided into four equal portions for profiling in Applied Biosystems 20×TAQMAN assays for analysis of RNA levels of PDGFRα, 18S, and GAPDH on the Fluidigm Biomark 48.48 Dynamic Array

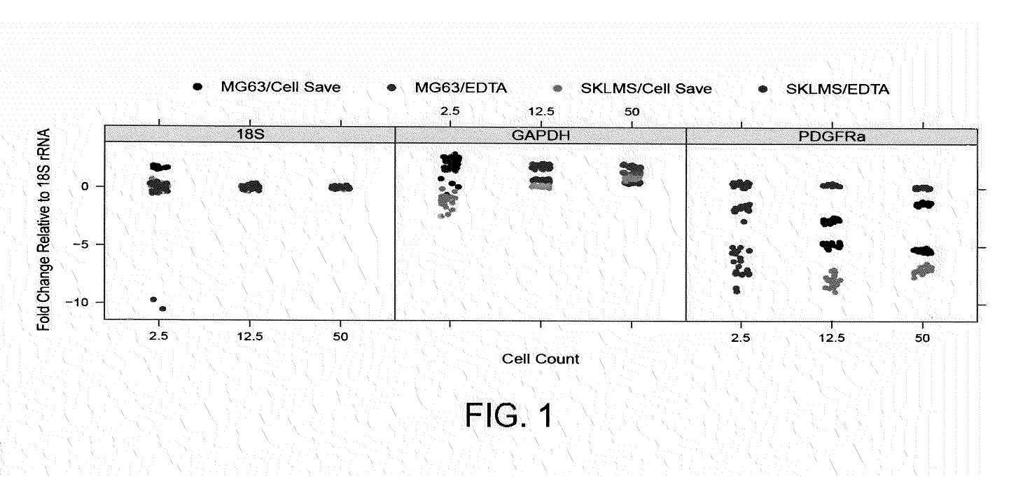

The PDGFRα mRNA expression levels in the cell line were calculated as described above and utilizing 18S ribosomal RNA as a control, with the result shown in FIG. 1. It can be seen that the expression of mRNA PDGRFα could be detected in both cells lines in quantities as low as that provided by 2.5 cells (corresponding to one fourth of a sample of 10 cells). The expressions of PDGFRα in both cells lines increased linearly to the 12.5 cell level, indicating that the method could reliably detect mRNA expression at the 12.5 cell level.

### 3. Sensitivity and Reliability of the Assay With a Leukocyte Background

PDGFRα expression levels were measured in different tumor cells which were spiked in CellSave tubes with a leukocyte background. Multiple 7.5 ml tubes of whole blood from healthy individuals were collected into CellSave tubes. Blood samples were processed in the CELLSEARCH System according to manufacturer's instructions. Quantities of 2, 5, and 10 cells from MG63 (higher expressor), SKLMS (medium expressor), and PC3M (lower expressor) cell lines, respectively, were spiked into these CellSave tubes containing background leukocytes. Samples were incubated with 100 μl proteinase K buffer mix and then 3001 Trizol LS was added. RNA was isolated from treated lysates using Zymo mini RNA spin columns. Superscript III cDNA synthesis utilizing random hexamer priming was performed. The resulting volume of pre-amplified cDNA was diluted and PDGFRα expression levels were determined by PDGFRα specific assays profiled on the Fluidigm Biomark 48.48 Dynamic Array following the manufacturer's established protocol. Threshold cycle (CT) values displayed are the averages of replicates. The results are shown below in Table 1.

TABLE 1

| | | PDGFRα | | |
|---|---|---|---|---|
| | Cell line | (CTs) | 18S (CTs) | GAPDH (CTs) |
| PDGFRα | MG63 (2 cells) | 25.78439 | 19.91005138 | 19.51856343 |
| higher | MG63 (5 cells) | 25.31323 | 20.37309099 | 20.02568288 |
| expressor | MG63 (10 cells) | 22.13929 | 19.71418329 | 18.42590449 |
| PDGFRα | SKLMS (2 cells) | undetected | 20.47170091 | 20.59105047 |
| medium | SKLMS (5 cells) | 27.47517 | 20.00873497 | 19.26960448 |
| expressor | SKLMS (10 cells) | 27.28056 | 19.55238196 | 18.95053002 |
| PDGFRα | PC3M (2 cells) | undetected | 20.85667449 | 21.58258632 |
| lower | PC3M (5 cells) | undetected | 20.84587066 | 20.90942977 |
| expressor | PC3M (10 cells) | 26.9 | 20.10632228 | 20.29019185 |

It can be seen that the methods disclosed herein could not only detect the expression of mRNA of PDGFRα at the 2 cell level in the higher expressor cell line MG63, but also could detect the mRNA of PDGFRα at the 10 cell level in the low expressor cell line PC3M, in each case with a background environment typically containing 1000 or more contaminating leukocytes pulled down from blood using the CELLSEARCH system.

### 4. Qualitative and Quantitative Measurement of Multiple Target Genes in CTC Samples from Cancer

Candidate epithelial cell markers, CTC markers, and drug targets were selected, and are listed below in Table 2.

TABLE 2

| | |
|---|---|
| Epithelial markers | MUC-1 |
| | EPCAM |
| | TACSTD2 |
| | MGB1 |
| | KRT19 |
| | KRT20 |
| Reported CTC marker | S100A16 |
| | AGR2 |
| | ASGR2 |
| Drug target | PDGFRα |
| | CEACAM5 |
| | EphA2 |
| | D114 |
| | EGFR |
| | HER2 |
| | HER3 |

It was desired to ascertain which of these candidate markers were expressed in leukocytes. Even in samples of enriched CTCs, leukocytes can be found in quantities of 1000 cells or more. In one aspect, genes selected for profiling are not expressed at significant levels in leukocytes, so that levels of expression in CTCs are not masked by expression in the leukocyte population in a sample.

To determine the background levels of the mRNA expression of these candidate genes in the contaminating leukocyte population, 7.5 ml of whole blood from 10 healthy individuals was collected in a CellSave tube. Samples were processed in the CELLSEARCH System according to the manufacturer's instructions. Following proteinase K treatment and Trizol addition, RNA was extracted, cDNA and pre-amplification reactions were carried out, and expression was assessed utilizing the Fluidigm Biomark using commercially available primers purchased from Applied Biosystems. No relative expression levels were analyzed. A CT value of <30 was considered positive; any CT exceeding this threshold was determined to be undetected. The results are shown in below in Table 3.

TABLE 3

| | | Expression in |
|---|---|---|
| Category | Genes | Leukocytes |
| Epithelial markers | MUC-1 | Positive |
| | EPCAM | Positive |
| | TACSTD2 | Positive |
| | MGB1 | Negative |
| | KRT19 | Positive |
| | KRT20 | Negative |
| Reported CTC marker | S100A16 | Positive |
| | AGR2 | Negative |
| | ASGR2 | Positive |
| Drug target | PDGFRα | Negative |
| | CEACAM5 | Negative |
| | EphA2 | Negative |
| | D114 | Negative |
| | EGFR | Positive |
| | HER2 | Positive |
| | HER3 | Negative |

As seen in Table 3, it was found that KRT20, MGB1, and AGR2 are three markers that are not expressed in leukocytes. As epithelial or tumor specific makers, they could be used as internal control genes to quantitatively measure the expression of the specific target genes from CTC. Furthermore, most of the evaluated drug targets (namely CEACAM5, Dll4, EphA2, Her3, and PDGFRα) do not have background expression in leukocytes, and are therefore also suitable for the assay.

DLL4 is expressed on endothelial cells and is not broadly expressed on tumor cells, but reported to be expressed on a subset of cancer cells that can be associated with cancer stem cells. If DLL4 were detected in a gene expression profile of a patient, then the patient would be a candidate for treatment using an anti-DLL4 biologic or corresponding therapeutic agent. Tumor expression of DLL4 would not be expected to be in a patient's archival tumor sample.

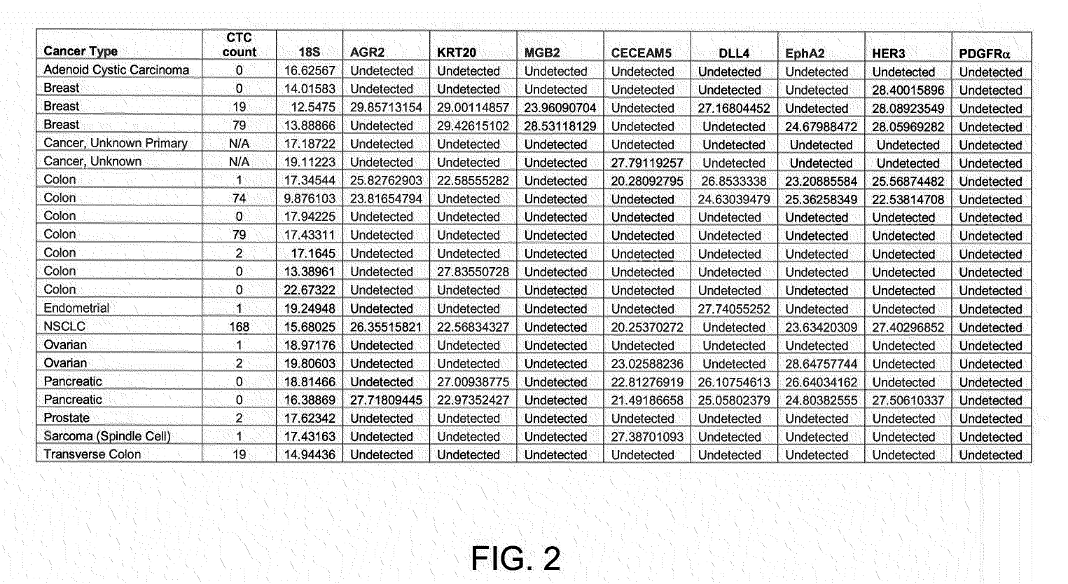

The assay as described herein was tested in CTC samples from cancer patients in order to profile specific gene expressions. Two samples of whole blood of 7.5 ml each from each of 22 cancer patients were collected in CellSave tubes. One of each pair of samples was used for testing with epithelial or tumor specific makers (KRT20, MGB1, and AGR2) and drug targets (CEACAM5, Dll4, EphA2, Ier3, and PDGRFα) and the other was used to obtain a CTC count. Samples were processed in the CELLSEARCH System according to manufacturer's instructions. The collected cells were treated with proteinase K buffer, RNA was isolated, and cDNA was generated, according to the process described above. The resulting cDNA was concentrated into a set volume utilizing magnetic beads. The cDNA of the target genes of interest was pre-amplified. The pre-amplified cDNA was then diluted and profiled on the Fluidigm Biomnark 48.48 Dynamic Array following the manufacturer's established protocol. The results are shown in FIG. 2.

Referring to FIG. 2, the CTC counts are known to underestimate the actual number of cells, therefore it is possible or even likely that in samples with a CTC count of zero do indeed contain CTCs. “N/A” in the CTC count column means the count is not available. The data in FIG. 2 is provided in the same CT units as other data herein. It was surprising and unexpected that several markers such as DLL4 could be found in circulating tumor cells.

KRT20 is expressed in CTCs across multiple tumor types and MGB1 is a breast cancer specific marker. KRT20, MGB1, and AGR2 are markers that are not expressed in leukocytes; therefore, the expression of these genes is attributable to the CTC population.

It was also surprising and unexpected that HER3 and CEA could be found in circulating tumor cells. Because HER3 expression can confer resistance to anti-HER2 treatment, in one aspect, if a circulating gene expression profile demonstrates the expression of HER3, a medical treatment directed against HER2 is avoided. See, for example, US20080317753, incorporated herein by reference.

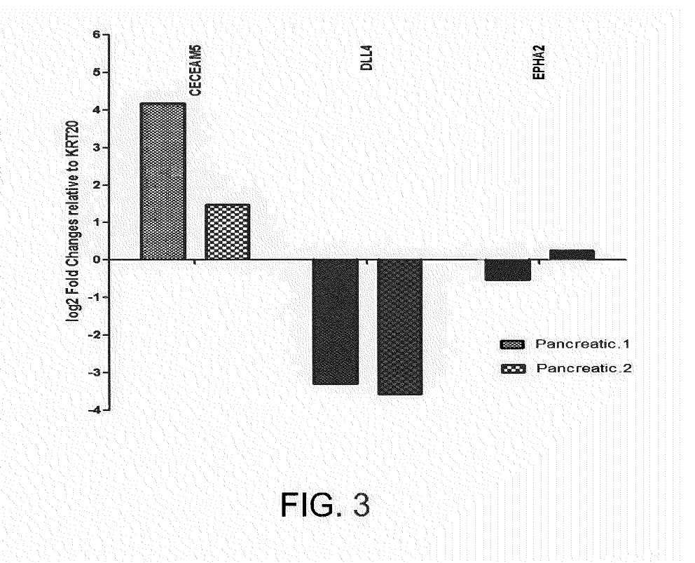

The above results indicated the method described herein could successfully generate a target gene expression profile in cancer patients. As seen in FIG. 3, quantitative analysis of target gene expression was also achieved in a sub-set of pancreatic cancer patients. KRT20 is a specific epithelial gene and consistently expressed in CTC samples in the sub-set of patients, and the expression level thereof in individual samples was used as reference to calculate the relative expression level of the other genes examined, such as CECAM5, DLL4, and EphA2.

The method described herein represents a sensitive, non-invasive technique that could be incorporated into various clinical trials and/or therapeutic regimes to assess the expression levels of specific therapeutic targets in patient tumors. Quantitative analysis of target gene expression is achievable with this method in situations wherein a CTC marker consistently expressed in a specific tumor type exists to serve as an internal control.

### REFERENCES

Each of the following references is incorporated herein by reference in its entirety.
Campos M, et al., Phenotypic and genetic characterization of circulating tumor cells by combining immunomagnetic selection and FICTION techniques. J Histochem Cytochem. 2008 July; 56(7):667-75.
Cristofanilli et al. Circulating Tumor Cells, Disease Progression, and Survival in Metastatic Breast Cancer. New England Journal of Medicine 351; 8 19 August 2004
Tewes M et al. Molecular profiling and predictive value of circulating tumor cells in patients with metastatic breast cancer: an option for monitoring response to breast cancer related therapies. Breast Cancer Res Treat. 2009 June; 15(3):581-90.
Helzer K T et al. Circulating tumor cells are transcriptionally similar to the primary tumor in a murine prostate model. Cancer Res. 2009 Oct. 1; 69(19):7860-6. Epub 2009 Sep. 29.
Spurgeon S L et al., High throughput gene expression measurement with real time PCR in a microfluidic dynamic array. PLoS One. 2008 Feb. 27; 3(2):e1662

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A rapid, sensitive method to obtain a gene expression profile from a target cell population in a blood sample, the method comprising:
(a) enriching the blood sample in target cells to obtain an enriched target cell sample;
(b) treating the enriched sample with a protease;
(c) extracting and purifying RNA from the sample;
(d) reverse transcribing the purified RNA to obtain a plurality of cDNAs; and
(e) analyzing the cDNAs with an amplification technique to obtain a target cell gene expression profile comprising expression levels of a plurality of mRNAs of interest,
wherein the target cell population represents less than 10% of the total cells in the blood sample.

2. The method of claim 1, wherein the plurality of mRNAs of interest comprises at least one mRNA not normally expressed in leukocytes.

3. The method of claim 1, wherein the target cells are circulating tumor cells.

4. The method of claim 1, wherein the target cells represents less than 5% of the cells in the blood sample.

5. The method of claim 1, wherein the target cells represents less than 1% of the cells in the blood sample.

6. The method of claim 1, wherein the target cells represents less than 0.5% of the cells in the blood sample.

7. The method of claim 1, wherein the target cells represents less than 0.1% of the cells in the blood sample.

8. The method of claim 1, wherein the cDNAs are concentrated in a set volume.

9. The method of claim 7, wherein the target cells are enriched using magnetic beads.

10. The method of claim 1, wherein the plurality of mRNAs of interest comprise at least one cDNA selected from the group consisting of DLL4, HER3, CEACAM5, KRT20, MGB1, and AGR2.

11. The method of claim 1, wherein the plurality of mRNAs of interest comprise at least one therapeutic target.

12. The method of claim 1, wherein the plurality of mRNAs of interest comprises an internal control comprising a marker consistently expressed in circulating tumor cells.

13. The method of claim 1, wherein none of the mRNAs of interest are normally expressed in leukocytes.

14. The method of claim 1, wherein the isolating the RNA comprises treating with a serine protease.

15. The method of claim 1, wherein the analyzing the cDNAs comprises performing real-time PCR.

16. The method of claim 1, further comprising performing a medical treatment on an individual based on the circulating tumor cell gene expression profile in a blood sample from the individual.

17. The method of claim 1, wherein the blood sample has been subjected to fixation.

18. The method of claim 1, further comprising a step of obtaining the blood sample from an individual.

19. A method of treating a patient having a tumor, the method comprising:
(a) obtaining a circulating gene expression profile from the patient by the method of claim 1, and
(b) performing a medical treatment on the patient based on the circulating tumor cell gene expression profile.

20-21. (canceled)

22. A kit comprising:
an antibody or functional fragment thereof specific for a cell surface marker found on target cells, and
primers adapted to amplify at least one cDNA, wherein the at least one cDNA comprises at least one cDNA not normally expressed in leukocytes.

23-25. (canceled)
